(19) United States
(12) Patent Application Publication
(10) Pub. No.: US 2025/0185904 A1
(43) Pub. Date: Jun. 12, 2025

(54) ENDOSCOPIC TUBING SET WITH COAXIAL ELASTOMERIC DIAPHRAGM

(71) Applicant: Boston Scientific Scimed, Inc., Maple Grove, MN (US)

(72) Inventors: Scott Edward Corbeil, Litchfield, NH (US); Colby Harris, Norfolk, MA (US); Nathan Thomas Cummings, Worcester, MA (US)

(73) Assignee: Boston Scientific Scimed, Inc., Maple Grove, MN (US)

(21) Appl. No.: 18/976,753

(22) Filed: Dec. 11, 2024

Related U.S. Application Data
(60) Provisional application No. 63/609,090, filed on Dec. 12, 2023.

(51) Int. Cl. A61B 1/12; A61B 1/00; A61B 1/005; A61B 1/015

(52) U.S. Cl. CPC A61B 1/126; A61B 1/00119; A61B 1/005; A61B 1/015

(57) ABSTRACT

Methods and systems for providing a flow of fluid to an endoscope. An illustrative container and tube set arranged and configured to couple to an endoscope may comprise a first container configured to contain a fluid and having a first port in fluid communication with a bottom portion thereof and a pressure vessel system. The pressure vessel system may include a housing defining a first cavity, a sleeve disposed within the first cavity of the housing and defining a second cavity between an inner surface of a wall of the housing and the sleeve, a first fluid inlet in selective fluid communication with the first cavity, a second fluid inlet in fluid communication with the second cavity, and a first fluid outlet in fluid communication with the first cavity.

### CROSS-REFERENCE TO RELATED APPLICATIONS

This application claims the benefit of U.S. Provisional Patent Application Ser. No. 63/609,090 filed on Dec. 12, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, having a clear field of vision is important to endoscope navigation and for clear visualization of anatomic structures during diagnostic and therapeutic procedures. In some cases, blood, tissue, fecal matter, mucus, or other bodily fluids or tissue may foul up the lens resulting in visual impairment. During endoscopic procedures, physicians may use a combination of air, irrigation, and lens wash as a means of flushing debris, cleaning optics, and insufflating the working lumen. An illustrative lens wash feature may shoot a jet of water from a nozzle at the distal end of the endoscope across the endoscope lens. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container and tube set arranged and configured to couple to an endoscope for use in an endoscopic procedure may comprise a first container configured to contain a fluid, the first container having a first port in fluid communication with a bottom portion thereof and a pressure vessel system. The pressure vessel system may comprise a housing defining a first cavity, a sleeve disposed within the first cavity of the housing and defining a second cavity between an inner surface of a wall of the housing and the sleeve, a first fluid inlet in selective fluid communication with the first cavity, a second fluid inlet in fluid communication with the second cavity, and a first fluid outlet in fluid communication with the first cavity.

Alternatively or additionally to any of the examples above, in another example, the container and tube may further comprise a first cap secured to a first end of the housing.

Alternatively or additionally to any of the examples above, in another example, the first fluid inlet may be formed in the first cap.

Alternatively or additionally to any of the examples above, in another example, the container and tube may further comprise a second cap secured to a second end of the housing.

Alternatively or additionally to any of the examples above, in another example, the first fluid outlet may be formed in the second cap.

Alternatively or additionally to any of the examples above, in another example, the second fluid inlet may be formed in the second cap.

Alternatively or additionally to any of the examples above, in another example, the container and tube may further comprise a first flow control member positioned adjacent to the first fluid inlet.

Alternatively or additionally to any of the examples above, in another example, the container and tube may further comprise a secondary housing fluidly coupled with the second fluid inlet, the secondary housing defining a lumen.

Alternatively or additionally to any of the examples above, in another example, the container and tube may further comprise a piston disposed within the secondary housing.

Alternatively or additionally to any of the examples above, in another example, the piston may be actuatable to selectively couple the second fluid inlet with the second cavity.

Alternatively or additionally to any of the examples above, in another example, the container and tube may further comprise a biasing member disposed between an end of the piston and an end of the secondary housing, the biasing member configured to bias the piston towards the second fluid inlet.

Alternatively or additionally to any of the examples above, in another example, the housing may be a rigid tubular member.

Alternatively or additionally to any of the examples above, in another example, the sleeve may be a flexible tubular member.

Alternatively or additionally to any of the examples above, in another example, the container and tube may further comprise a slot extending through a sidewall of the housing, the slot may be configured to be in fluid communication with the second fluid inlet.

Alternatively or additionally to any of the examples above, in another example, a volume of second cavity may be configured to selectively increase to increase a pressure of the first cavity.

Alternatively or additionally to any of the examples above, in another example, the container and tube may further comprise a flow control mechanism fluidly coupled to the first fluid outlet.

Alternatively or additionally to any of the examples above, in another example, the container and tube may further comprise a vent in one of the first or second caps, the vent fluidly coupling the first cavity with an atmosphere outside the system.

Alternatively or additionally to any of the examples above, in another example, the first fluid outlet and the second fluid inlet may be formed in the second cap.

In another example, a container and tube set arranged and configured to couple to an endoscope for use in an endoscopic procedure may comprise a first container configured to contain a fluid, the first container having a first port in fluid communication with a bottom portion thereof and a pressure vessel system. The pressure vessel system may comprise a generally tubular housing extending from a first end to a second end and defining a first cavity, a first cap coupled with the first end of the generally tubular housing, a second cap coupled with the second end of the generally tubular housing, a sleeve extending from a first end to a second end and disposed within the first cavity of the housing and defining a second cavity between an inner surface of a wall of the housing and the sleeve, a first fluid inlet in selective fluid communication with the first cavity, a second fluid inlet in fluid communication with the second cavity, and a first fluid outlet in fluid communication with the first cavity. The first end of the sleeve may be positioned between an outer surface of the generally tubular housing and the first cap and the second end of the sleeve may be positioned between the outer surface of the generally tubular housing and the second cap.

Alternatively or additionally to any of the examples above, in another example, a volume of the second cavity may be configured to selectively increase to increase a pressure of the first cavity.

In another example, a container and tube set arranged and configured to couple to an endoscope for use in an endoscopic procedure may comprise a first container configured to contain a fluid, the first container having a first port in fluid communication with a bottom portion thereof and a pressure vessel system. The pressure vessel system may comprise a generally tubular housing extending from a first end to a second end and defining a first cavity, a first cap coupled with the first end of the generally tubular housing, a second cap coupled with the second end of the generally tubular housing, a secondary housing extending from a lateral side of the second cap, the secondary housing defining a lumen, a piston disposed within the lumen of the secondary housing, a sleeve extending from a first end to a second end and disposed within the first cavity of the housing and defining a second cavity between an inner surface of a wall of the housing and the sleeve, a first fluid inlet in selective fluid communication with the first cavity, a second fluid inlet in fluid communication with the second cavity, and a first fluid outlet in fluid communication with the first cavity.

Alternatively or additionally to any of the examples above, in another example, the piston may be actuatable to selectively couple the second fluid inlet with the second cavity.

Alternatively or additionally to any of the examples above, in another example, the container and tube may further comprise a biasing member disposed between an end of the piston and an end of the secondary housing, the biasing member configured to bias the piston towards the second fluid inlet.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 is a front view of an illustrative pressure vessel system;

FIG. 5 is a side view of the illustrative pressure vessel system of FIG. 4;

FIG. 6 is a cross-sectional view of the illustrative pressure vessel system, taken at line 6-6 of FIG. 5;

FIG. 7 is a cross-sectional view of the illustrative pressure vessel system in a pressurized configuration;

FIG. 8 is an enlarged view of a portion of the illustrative pressure vessel system taken at Detail A of FIG. 7;

FIG. 9 is a front view of another illustrative pressure vessel system;

FIG. 10 is a cross-sectional view of the illustrative pressure vessel system of FIG. 9 in an unpressurized configuration;

FIG. 11 is cross-sectional view of the second end region of the illustrative pressure vessel system in an unpressurized configuration;

FIG. 12 is a cross-sectional view of the illustrative pressure vessel system of FIG. 9 in a pressurized configuration; and

FIG. 13 is cross-sectional view of the second end region of the illustrative pressure vessel system in a pressurized configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/of” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Additionally, current water bottle and tube sets may leak if not threaded properly. Finally, the water bottle may require a level surface to be placed properly which, in an endoscopy suite, may be at a premium. Disclosed herein are methods and systems to reduce or eliminate the need to disconnect the tube set and use a second bottle.

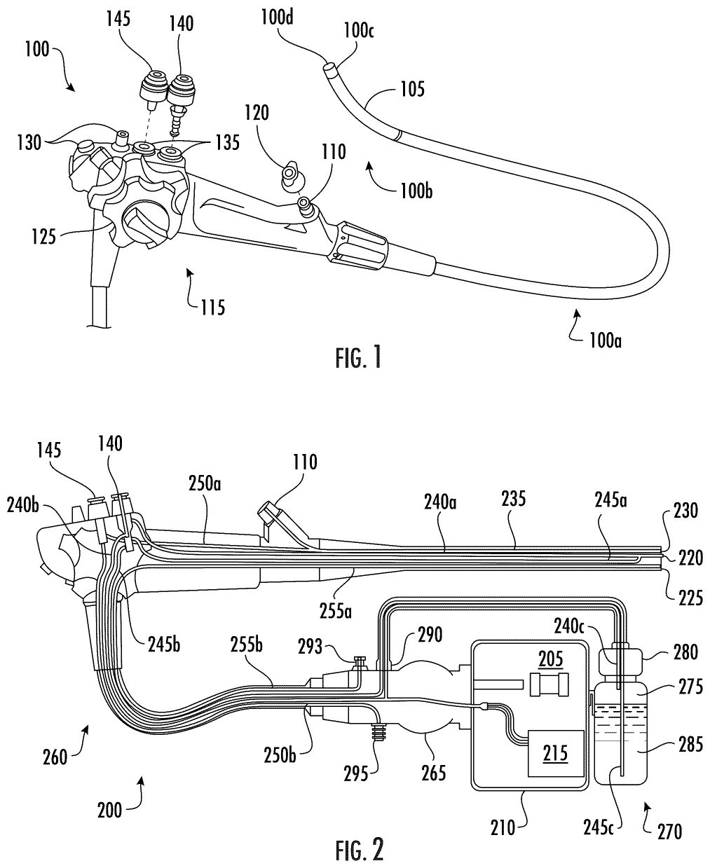

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

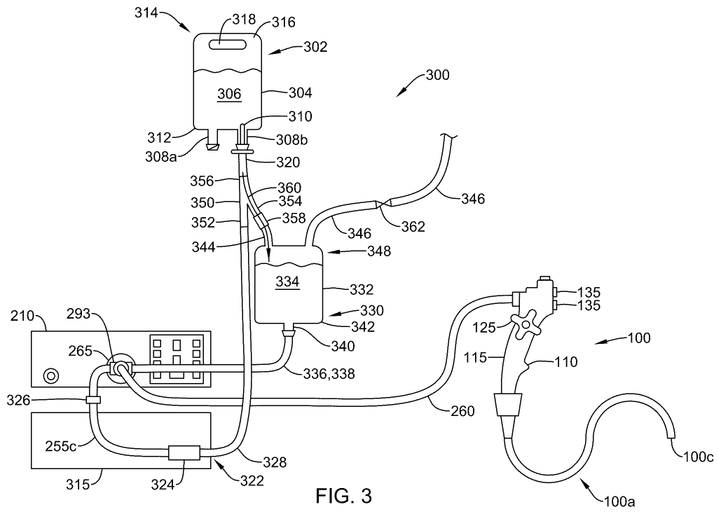

FIG. 3 depicts a schematic view of another illustrative endoscopic system 300 which may reduce the number of water reservoir changes and/or reduce opportunities for contamination during replacement of the water reservoir(s). The system 300 may include a number of advantages over the current bottle system described above. The system 300 may include components similar to the endoscope and endoscope systems described with regard to FIGS. 1-2; however, not all features may be described or shown here.

Generally, the system 300 may include a first reservoir 302 and a second reservoir 330. The first reservoir 302 may be configured to supply water or fluid for both irrigation (e.g., via the first reservoir 302) and lens wash (e.g., via the second reservoir 330). This may allow a single fluid source to be used to provide fluid for both irrigation and lens wash. While not explicitly shown, the reservoirs 302, 330 may include printed lines, numbers, or other visual indicia to allow a user to easily determine how much fluid is left in the reservoirs 302, 330.

The first reservoir 302 may include a first container 304 configured to hold a first volume of fluid 306. In the illustrated embodiment, the first container 304 is fluidly coupled to the upstream irrigation supply tubing 328 and is configured to provide fluid for irrigation to the endoscope 100. Generally, the irrigation supply tubing 328 may be a water or fluid supply line or tube for supplying water or other fluid to an endoscope. Additionally, the first container 304 may be selectively fluidly coupled to a second fluid reservoir 330. The second reservoir 330 may include a second container 332 configured to hold a second volume of fluid 334. In the illustrated embodiment, the second container 332 is fluidly coupled to the gas and lens wash supply tubing 336, 338 and is configured to provide fluid for lens wash to the endoscope 100. Generally, the lens wash supply tubing 338 may be a water or fluid supply line or tube for supplying water or other fluid to an endoscope. The gas and lens wash supply tubing 336, 338 may be coaxially arranged. For example, the gas supply tubing 336 may define a lumen that is sufficiently large in diameter to encompass a smaller diameter lens wash tubing 338, coaxially received within the gas supply tubing 336, as well as provide air to the water source in an annular space surrounding the lens wash tubing 338 to pressurize the second reservoir 330. The lens wash supply tubing 338 may be configured to exit the lumen defined by the coaxial gas supply tubing 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 336, 338 may be arranged in a side-by-side arrangement.

The first and second containers 304, 332 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 304, 332 may be entirely translucent, entirely opaque, or combinations thereof. In some cases, the first and second containers 304, 332 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 304, 332 may be variable. For example, the volume of the first container 304 and/or the second container 332 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 302, 330 may be pre-filled (e.g., prior to entering the procedure suite or at the time of manufacturing) with water or other fluid. In some cases, the clinician may select the reservoir(s) 302, 330 from a plurality of differently sized available reservoirs based on the number and/or types of procedures expected for a typical or the specific day. In the illustrated embodiment, the first reservoir 302 may supply fluid to the second reservoir 330. By selecting a first reservoir 302 having a volume large enough to accommodate an entire day of procedures, the need for replacing the sterile fluid source (e.g., the first reservoir 302) may be reduced or eliminated. In some cases, the first reservoir 302 may be used to periodically refill the second reservoir 330. Thus, the volume of the first reservoir 302 may be greater than the volume of the second reservoir 330, although this is not required. It is further contemplated that, in some embodiments, one or both of the first or second reservoirs 302, 330 may be a rigid bottle.

It is contemplated that flexible bags may utilize less plastic (or other material) than a bottle designed to hold a similar amount of fluid. Thus, the use of a flexible bag as a fluid reservoir 302, 330 may increase the level of environmental sustainability of the system 300. For example, if the user sets up the system with a 3000 mL (3 liter) bag reservoir 302 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a fluid or water supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water supply tube) 328 while another port 308a may be configured to allow the user to add additives to the fluid 306. In other examples, the upstream irrigation tubing 328 may be coupled to the first port 308a while the second reservoir 330 is in fluid communication with the second port 308b. While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328, branched connector 350, and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material to the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of the upstream irrigation supply tube 328. The upstream irrigation supply tube 328 extends from a second end region 322 external to the container 304 and positioned within a pump head 324 of the peristaltic irrigation pump 315 to a first end 320. The first end 320 of the upstream irrigation supply tube 328 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 302 may be inserted into a compression sleeve. When irrigation fluid is desired, the compression sleeve may be activated to exert pressure on an outer surface of the reservoir 302 and to provide the required pressure to perform irrigation at the distal end of the endoscope 100. In another embodiment the reservoir 302 may be inserted into a compression sleeve which applies constant pressure to the reservoir 302 with a flow switch positioned along irrigation supply tubing 328 to provide binary control of irrigation flow.

The second reservoir 330 may further include one or more ports 340, such as, but not limited to a spike port or a septum port, extending from and in selective fluid communication with an interior of the second container 332. The port 340 may be formed as a monolithic structure with the second container 332. The port 340 may be a generally tubular structure with the port 340 defining a lumen extending therethrough. The lumen of the port 340 may be configured to selectively fluidly couple the interior of the second container 332 with another component, such as, but not limited to, fluid/water/gas supply tube(s). In some cases, the port 340 may be configured to be coupled to the gas and lens wash supply tubing 336, 338. In some embodiments, the port 340 may be positioned adjacent to a bottom end 342 of the second reservoir 330. However, this is not required. The port 340 may be positioned in other locations, as desired. If the port 340 is positioned at a location other than the bottom end 342 of the second container 332, a dip tube or tube extension may be required (e.g., coupled to the lens wash supply tubing 338) to access the fluid at the bottom of the second container 332. While the second reservoir 330 is illustrated as including one port 340, the second reservoir 330 may include more than one port, as desired.

While not explicitly shown, the port 340 may include a removable cap or seal configured to form a fluid tight seal with the port 340. The removable cap or seal may help to maintain the sterility of the port 340. The removable cap or seal may be coupled to a free end of the port 340 using a number of different techniques. For example, the cap or seal may be coupled to the port 340 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 340. Once the cap or seal has been removed, the port 340 may be pierced with a spike tip or spike port adaptor (not explicitly shown) that is coupled to the gas and lens wash supply tubing 336, 338. For example, in addition to the removable cap or seal, the port 340 may include an internal seal disposed within a lumen of the port 340 that may be punctured or pierced by the spike port adaptor. The internal seal may be configured to prevent fluid 334 from leaking from the second container 332 prior to the spike port adaptor being inserted into the port 340. In some embodiments, the internal seal may be self-sealing such that upon removal of the spike port adaptor fluid is prevented from leaking from the port 340. The outer surface of the spike port adaptor may form an interference fit with the inner surface of the port 340. The fit and/or coupling between the spike port adaptor and the port 340 may be sufficient to remain in place when the gas and fluid supply tubing 336, 338 and/or other tubing sets are coupled to the spike port adaptor. It is contemplated that the spike port adaptor may be inserted into the port 340 utilizing universally used aseptic techniques such as those used with IV fluid bags. This may help reduce infection risk by maintaining sterile components, not introducing contaminants into the fluid 334, etc. It is further contemplated that additives may be added to the fluid 334 using similar aseptic techniques via the port 340, if so desired. In some cases, other coupling mechanisms may be used as desired to couple the gas and lens wash supply tubing 336, 338 to the port 340. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 336 extends from a second end external to the second container 332 to the port 340. The gas supply tubing 336 may extend into the interior of the second container 332 and terminate within a reservoir gap (e.g., above the level of the fluid 334). However, in some cases, the gas supply tubing 336 may terminate within the fluid 334. A lumen extends through the gas supply tubing 336 for receiving a flow of air and/or gas therethrough. The lumen of the gas supply tubing 336 may be in operative fluid communication with a top portion of the interior of the second container 332. The lens wash supply tubing 338 extends from a second end external to the second reservoir 330 to a first end in fluid communication with a bottom portion 342 of the second container 332. In some embodiments, the lens wash supply tubing 338 may terminate at the port 340. A lumen extends through the lens wash supply tubing 338 for receiving a flow of fluid therethrough. The lumen of the lens wash supply 338 is in selective operative fluid communication with a bottom portion 342 of the second container 332. In the illustrated embodiment, the gas supply tubing 336 and the lens wash supply tubing 338 may couple to the second container 332 through a single or common opening (e.g., port 340). For example, the gas supply tubing 336 and the lens wash supply tubing 338 may be coaxially arranged. However, this is not required. In some cases, the gas supply tubing 336 and the lens wash supply tubing 338 may extend in a side-by-side arrangement or may be separately connected to the second container 332 in different locations.

The second container 332 may further include a first fluid inlet 344 and a second fluid inlet 346. While the first and second fluid inlets 344, 346 are illustrated as being adjacent to or extending from a top portion 348 of the second container 332, the first and/or second fluid inlets 344, 346 may be positioned at other locations about the second container 332, as desired. In some embodiments, the first and/or second fluid inlets 344, 346 may be tubular members formed as a single monolithic structure with the second container 332. In other embodiments, the first and/or second fluid inlets 344, 346 may include tubular components releasably coupled to ports (similar in form and function to port 340) formed in or with the container 332.

The first fluid inlet 344 may be in selective fluid communication with the first reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the first reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port adaptor 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the first fluid inlet 344 of the second reservoir 330. A flow control mechanism, such as, but not limited to, a one-way valve 358 may be positioned between the second fluid outlet of the second outlet leg 354 and the first fluid inlet 344 of the second reservoir 330 to selectively fluidly couple the second container 332 with the first container 304. The one-way valve 358 may be configured to be opened to allow fluid to selectively pass from the first reservoir 302 to the second reservoir 330 while preventing fluid (e.g., gas, water, or other fluid) from exiting the second container 332 and entering the irrigation supply tubing 328 and/or the first container 304. In some embodiments, the one-way valve 358 may be replaced with a clamp which may compress the first fluid inlet 344 to selectively fluidly isolate the second container 332 from the first container 304 and removed to selectively couple the second container 332 with the first container 304. In yet other embodiments, the one-way valve 358 may be replaced with a spring-loaded valve, a stopcock, or other two-way valve. When it is desired to add fluid to the second reservoir 330 from the first reservoir 302, the one-way valve 358 (or other flow control mechanism) may be opened or released. Fluid may then be at least partially diverted from the irrigation supply tubing 328 through the second outlet leg 354 of the branched connector 350 and into the second container 332 along flow path 360. Fluid may be added to the second container 332 while the irrigation pump 315 is running or while the irrigation pump 315 is idle, as desired.

The second fluid inlet (or gas supply tube) 346 of the second container 332 may be an alternative gas supply tubing configured to be coupled to an alternative gas supply (e.g., CO₂ hospital house gas source). The second fluid inlet 346 may extend from a second end external to the second container 332 to a first end coupled to the second container 332. The alternative gas supply may be used to pressurize the second container 332 to supply lens wash to the endoscope 100 and/or to provide insufflation. A lumen extends through the second fluid inlet 346 for receiving a flow of gas therethrough. The lumen of the second fluid inlet 346 is in operative fluid communication with a top portion of the second container 332. The flow of the CO₂ through the system 300 may be similar to that described above. For example, in the neutral state, CO₂ gas flows through the second fluid inlet 346 into the second container 332, up the gas supply tubing 336 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 346 into the second container 332, up the gas supply tubing 336 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 330 is maintained by delivering gas through the second fluid inlet 346. It is contemplated that the one-way valve 358 is in the closed configuration during delivery of the CO₂ gas to allow the container 332 to pressurize. In some instances, the one-way valve 358 may be configured to close without user intervention in response to the delivery of CO₂ to the second container 332. In some embodiments, the system 300 may include a branched connector (such as, but not limited to a “Y” or “T” connector) at the second fluid inlet 346 to allow either air or CO₂ to be used for pressurization or insufflation. It is further contemplated that the second fluid inlet 346 may include a pressure relief valve 362, such as, but not limited to, a 3-way stopcock, a clamp, or a spring-loaded valve, to vent pressure within the second container 332 and/or to block a flow of pressurized gas to the second container 332 during refilling of the second container 332, during procedure change-overs, and/or during equipment change-overs.

It is contemplated that the use of a flexible bag in place of a rigid bottle for the second reservoir 330 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 332 is fluidly isolated from the first container 304 when the one-way valve 358 is closed, it is contemplated that the clinician may replace the first reservoir 302 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Further, the system 300 may remain largely closed as the first reservoir 302 is changed out.

In some cases, it may be desirable to reduce the number of features that need to be managed during a procedure. For example, it may be desirable to provide lens wash without a separate system or a secondary fluid storage vessel. This may improve the user interface and/or reduce the amount of space needed for the system. In some cases, a pressure vessel system may be in selective fluid communication with a fluid source, such as, but not limited to, the first reservoir 302. The pressure vessel system may be filled with an amount of fluid sufficient to perform a lens wash operation prior to actuation of the gas/water valve 140. In some cases, the pressure vessel system may be filled in response to a user input. In other cases, the pressure vessel system may be filled without user input. In either case, the user need not monitor the fluid level in the pressure vessel system or replace the pressure vessel system.

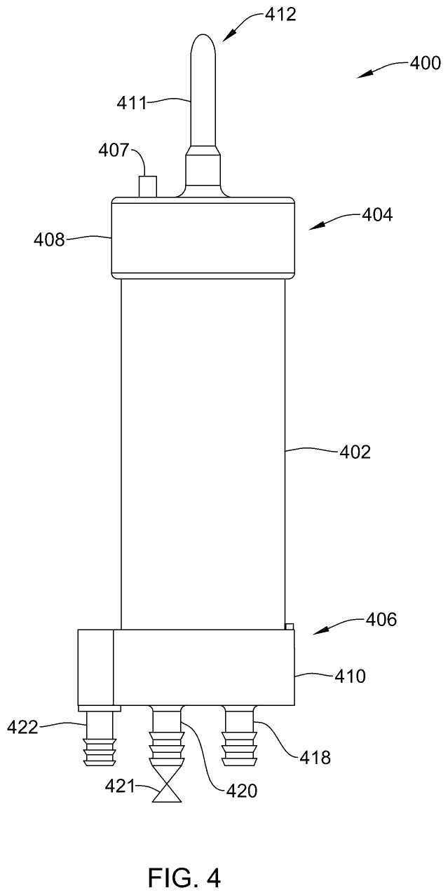

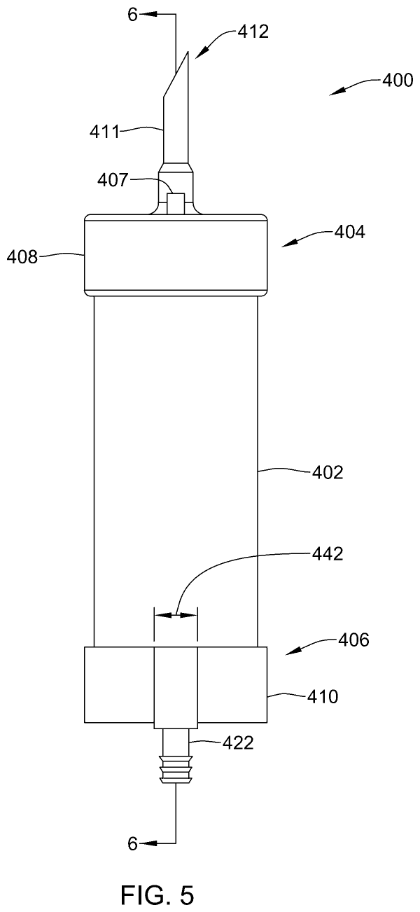

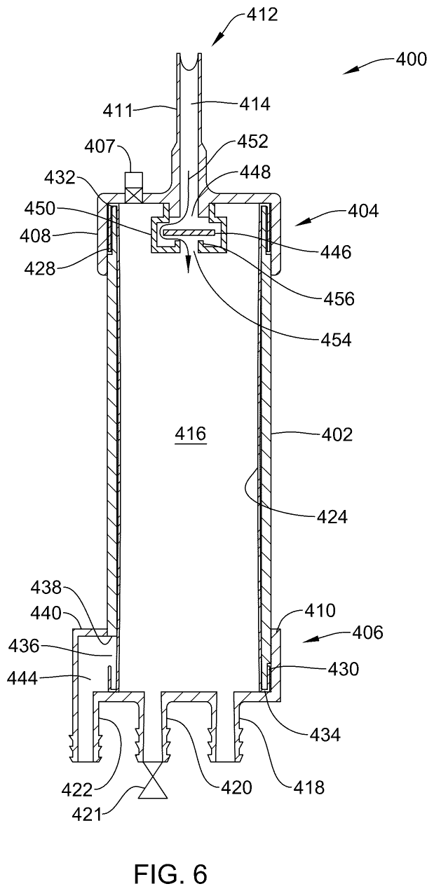

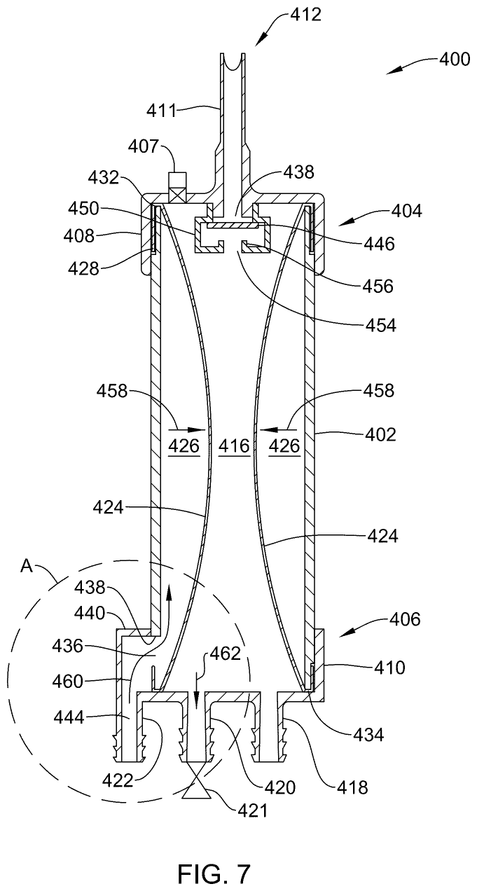

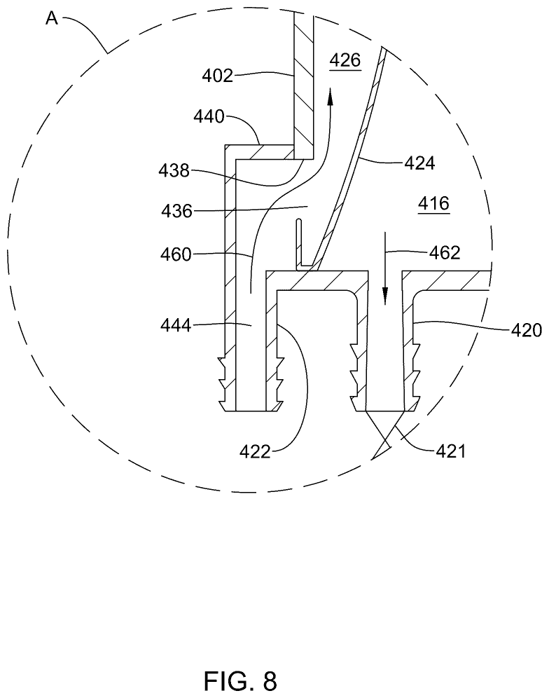

An illustrative pressure vessel system 400 that may be used in place of the secondary fluid reservoir 330 of FIG. 3 is shown in FIGS. 4-8. FIG. 4 is a front view of the illustrative pressure vessel system 400. FIG. 5 is a side view of the illustrative pressure vessel system 400. FIG. 6 is a cross-sectional view of the illustrative pressure vessel system 400, taken at line 6-6 of FIG. 5. FIG. 7 is a cross-sectional view of the illustrative pressure vessel system 400 in a pressurized configuration. FIG. 8 is an enlarged view of a portion of the illustrative pressure vessel system 400 taken at Detail A of FIG. 7. The pressure vessel system 400 may be configured to be fluidly and/or mechanically coupled to a fluid source, such as the first fluid reservoir 302 of FIG. 3. Generally, the system 400 may include a tubular housing 402 extending from a first end region 404 to a second end region 406. In some embodiments, the housing 402 may be formed from a rigid material configured to withstand the pressure required to supply the lens wash fluid. For example, the air pump 215 may be configured to supply pressurized air having a pressure in the range of about 7 to about 7.25 pounds per square inch (psi) (48.3 to about 50.0 kilopascals). Other pressure ranges may be used as desired. In some examples, the housing 402 may be formed from rigid materials such as metal, plastic, composite materials, or the like. In yet other examples, the housing 402 may be formed from a semi-rigid material.

A first lumen or cavity 416 may extend from the first end region 404 to the second end region 406 of the housing 402. In a first configuration (FIG. 6), when the gas/water valve 140 is in the neutral or first position (e.g., when lens wash is not being called for), the first cavity 416 may have a volume in the range of about 90-110 cubic centimeters (cc). The volume of the first cavity 416 may be reduced when lens wash is activated (FIG. 7). It is contemplated that the volume of the first cavity 416 in the first configuration may be less than 90 cc or greater than 110 cc depending on the application.

A first cap 408 may be releasably or fixedly secured to the first end region 404 and a second cap 410 may be releasably or fixedly secured to the second end region 406. The first and second caps 408, 410 may be configured to seal a first end opening and a second end opening of the housing 402, respectively. The first and second caps 408, 410 may include one or more fluid paths to selectively fluidly couple a fluid source with an endoscope 100, as will be described in more detail herein.

The first cap 408 may be generally tubular having a first generally closed end and a second end defining an opening. The opening may be sized such that the first cap 408 may be positioned over the first end region 404 of the housing 402. It is contemplated that the first cap 408 may be fixedly or releasably secured to the housing 402 in a fluid tight manner. Some illustrative coupling mechanisms may include, but are not limited to, threaded engagements, snap fits, friction fits, quick connect style couplers, adhesive or solvent bonding, thermal bonding, or the like.

The first cap 408 may include a first fluid inlet 411 extending from the first end thereof. In some embodiments, the first fluid inlet 411 may be a spike port adaptor 412. The spike port adaptor 412 may be received within the port of a fluid source. For example, the spike port adaptor 412 may be inserted directly into one of the ports 308a, 308b of the first fluid reservoir 302 of the system 300 of FIG. 3. Alternatively, in the system 300 of FIG. 3, the second outlet leg 354 of the branched connector 350 may include a spike port, similar in form and function to the ports 308a, 308b described herein, configured to receive the spike port adaptor 412. The spike port adaptor 412 may define a lumen 414 configured to fluidly couple the first fluid reservoir 302, or other fluid source, with the first cavity 416 of the housing 402 and ultimately with the lens wash supply tubing and/or irrigation supply tubing. The spike port adaptor 412 may be formed as a single monolithic structure with the first cap 408 or may be formed as a separate component subsequently coupled to the first cap 408.

In some embodiments, the first cap 408 may include a manual vent 407, such as a valve, stopcock, or the like. The manual vent 407 fluidly couples the first cavity 416 to an atmosphere outside of or exterior to the system 400 to allow the user to vent air from the first cavity 416 during the initial setup of the endoscope system. For example, air may be purged and/or displaced from the first cavity 416 via the vent 407 to fully fill the inner cavity 416 with fluid from the water source (e.g., fluid reservoir 302). It is contemplated that the manual vent 407 may be positioned at other locations within the pressure vessel system 400, as desired. For example, the manual vent 407 may extend through the second cap 410, through the housing 402, etc.

The second cap 410 may be generally tubular having a first end defining an opening and a second generally closed end. The opening may be sized such that the second cap 410 may be positioned over the second end region 406 of the housing 402. It is contemplated that the second cap 410 may be fixedly or releasably secured to the housing 402 in a fluid tight manner. Some illustrative coupling mechanisms may include, but are not limited to, threaded engagements, snap fits, friction fits, quick connect style couplers, adhesive or solvent bonding, thermal bonding, or the like.

The second cap 410 may include a first fluid outlet 418, a second fluid outlet 420, and a second fluid inlet 422 extending from the second end thereof. In some embodiments, the second fluid inlet 422 may extend along or otherwise be coupled to a lateral side of the second cap 410. However, this is not required. In some embodiments, the second inlet 422 and/or outlets 418, 420 may be tubular ports or openings in the second cap 410 configured to selectively fluidly couple the housing 402 with another component, such as, but not limited to, a fluid or water supply tube and/or a gas supply tube. For example, the first fluid outlet 418 may be configured to be coupled with an irrigation supply tubing (similar in form and function to irrigation supply tubing 328 described above) and the second fluid outlet 420 may be configured to be coupled with a lens wash supply tubing (similar in form and function to the lens wash supply tubing 338 described above. The second fluid inlet 422 may be configured to be coupled with a gas supply tubing or an alternative gas supply tubing (similar in form and function to the gas supply tubing 336 or alternative gas supply tubing 346 described above). The second fluid inlet 422 and/or outlets 418, 420 may include hose barbs or other features configured to secure the fluid or water supply tube and/or a gas supply tube thereto.

While the first and second fluid inlets 411, 422 and the first and second fluid outlets 418, 420 are shown and described as being a part of the first and second caps 408, 410. The fluid inlets 411, 422 and/or fluid outlets 418, 420 may be formed in other parts of the pressure vessel system 400, as desired. For example, one or more of the fluid inlets 411, 422 and/or fluid outlets 418, 420 may be formed in the housing 402 of the pressure vessel system 400. It is further contemplated that while the fluid inlets 411, 422 and fluid outlets 418, 420 are shown and described as extending or protruding from the first and second caps 408, 410, the fluid inlets 411, 422 and/or fluid outlets 418, 420 may be formed as recesses or cavities. Further, as described above, the fluid inlets 411, 422 and/or fluid outlets 418, 420 may be formed as separate structures from the caps 408, 410 (or other portions of the pressure vessel system 400) and may be subsequently coupled with the caps 408, 410 (or other portions of the pressure vessel system 400) using solvent bonding, adhesives, mechanical couplings, or the like.

Referring more particularly to FIG. 6, the pressure vessel system 400 may further include an elastomeric or flexible sleeve 424 disposed within the first cavity 416 of the housing 402. The flexible sleeve 424 may have a generally tubular configuration and may be positioned along the inner surface of the housing 402 (e.g., within the first cavity 416). The flexible sleeve 424 may be free from attachment to the housing 402 along at least a portion of the length of the flexible sleeve 424 to define a variable volume space or second cavity 426 between the inner surface of the wall of the housing 402 and the flexible sleeve 424. The flexible sleeve 424 may be formed from a flexible or deformable material that can be stretched and subsequently return to an original configuration. For example, in response to an increase in air/gas volume in the space or second cavity 426 between the wall of the housing 402 and the flexible sleeve 424, the flexible sleeve 424 may extend radially inwards reducing the volume of the first cavity 416. As air is evacuated the second cavity 426 and the volume thereof reduces, the flexible sleeve 424 may return to its original configuration along the inner surface of the wall of the housing 402. In some embodiments, the flexible sleeve 424 may be formed from a thin wall silicone, rubber, thermoplastic elastomer (TPE), or other elastomeric material which can be stretched to create tensile loading and capable of re-coiling after being strained in the plastic deformation region of the stress-strain curve. The flexible sleeve 424 may take many forms. In the illustrative example, the flexible sleeve 424 may be cylindrical and positioned along an inside wall of the tubular housing 402, thus allowing pressurization from all sides and facilitating a larger displacement efficiency. In other examples, the flexible sleeve 424 may extend less than 360° about an inner circumference of the tubular housing 402. When the flexible sleeve 424 extends less than 360°, the lateral edges of the flexible sleeve 424 may be secured along the length of the sleeve 424 to the inner surface of the housing 402 to form an air-tight seal.

The flexible sleeve 424 may extend from a first end 428 to a second end 430. The flexible sleeve 424 may have a length greater than a length of the housing 402. In some examples, the first end 428 of the flexible sleeve 424 may extend beyond a first end 432 of the housing 402 and fold over to extend along an outer surface of the housing 402. The first end 428 of the flexible sleeve 424 may be secured between an inner surface of the first cap 408 and the outer surface of the housing 402. In some examples, the second end 430 of the flexible sleeve 424 may extend beyond a second end 434 of the housing 402 and fold over to extend along an outer surface of the housing 402. The second end 430 of the flexible sleeve 424 may be secured between an inner surface of the second cap 410 and the outer surface of the housing 402. The wall thickness of the housing 402 may be reduced or thinned for a length adjacent to the first end 432 and/or the second end 434 of the housing 402. This may allow the flexible sleeve 424 to be more easily mechanically captured between the caps 408, 410 and the outer surface of the housing 402. However, this is not required. In some embodiments, the housing 402 may have a uniform wall thickness along an entire length thereof. In some cases, the flexible sleeve 424 may be additionally or alternatively coupled to the housing 402 using a number of techniques such as adhesive or solvent bonding, thermal bonding, and the like. In another embodiment, the flexible sleeve 524 may transition from a thin wall to a thick ring at one or both ends 428, 430. The thick ring(s) may function as a gasket between the rigid housing 402 and one or both caps 408, 410. It is further contemplated that the flexible sleeve 424 may have a length that is less than a length of the housing 402. For example, one or more of the ends 428, 430 of the flexible sleeve 424 may be secured at a location between the first and second end 432, 434 of the housing 402.

Referring additionally to FIG. 8, the housing 402 may further include a slot or opening 436 extending from the second end 434 thereof towards the first end 432 thereof. In some cases, the slot or opening 436 may be spaced from the second end 434 of the housing 402. An end 438 of the slot 436 may be positioned between an enclosed end 440 of the second fluid inlet 422 and the second end 434 of the housing 402. Further, the slot 436 may have a width less than a width 442 of the second fluid inlet 422 such that the slot 436 is in fluid communication with a lumen 444 of the second fluid inlet 422 but does not extend beyond the lumen 444 of the second fluid inlet 422. While not explicitly shown, in some examples, the housing 402 and the second cap 410 may include mating alignment features (such as, but not limited to, a tab and slot or visual indicia) configured to facilitate assembly of the second cap 410 with the housing 402 such that the second fluid inlet 422 aligns with the slot 436. The slot 436 may fluidly couple a lumen 444 of the second fluid inlet 422 with the second cavity 426. Thus, pressurized air/gas may flow from the air pump 215 or alternative gas source through the second fluid inlet 422 in the second cap 410 and into the second cavity 426 to fill/pressurize the second cavity 426.

A flow control member 446 may be positioned within the first cavity 416 of the housing 402 adjacent to the first fluid inlet 411. The flow control member 446 may be configured to selectively block or occlude an opening 448 of the first fluid inlet 411. When the flow control member 446 is disposed against or over the opening 448 of the first fluid inlet 411, fluid is prevented from flowing from the fluid source (e.g., first reservoir 302) into the first cavity 416 of the housing 402 (see, for example, FIG. 7) and from flowing from the first cavity 416 of the housing 402 into the fluid source. In some cases, the flow control member 446 may be a floating stopper selectively held against the first end opening 448 of the first fluid inlet 411. The flow control member 446 may be disposed against or over the first end opening 448 of the first fluid inlet 411 by an increase in the pressure in the first cavity 416 within the housing 402. When the pressure is vented (e.g., by releasing the gas/water valve 140), the pressure in the first cavity 416 of the housing 402 may decrease allowing the flow control member 446 to be displaced away from first end opening 448 of the first fluid inlet 411 such that fluid can flow into the first cavity 416 of the housing 402, as shown at arrow 452. A stop mechanism 450 may be positioned adjacent to the flow control member 446 to prevent the flow control member 446 from dropping to a bottom of the housing 402. In some examples, the flow control member 446 may contact an upper edge of the stop mechanism 450 to stop a flow of fluid through the first fluid inlet 411. The stop mechanism 450 may include one or more openings 454 that are smaller than the flow control member 446 to allow fluid to pass while maintaining the flow control member 446 adjacent to the opening 448 of the first fluid inlet 411. The flow control member 446 may be spaced from the opening 454 by one or more circumferentially spaced ribs 456 extending from the stop mechanism 450. The one or more circumferentially spaced ribs 456 may have gaps or openings therebetween to allow fluid to pass between the flow control member 446 and the stop mechanism 450. In other examples, the flow control member 446 may be a one-way valve that allows fluid to flow into the first cavity 416 of the housing 402 when the pressure of the first cavity 416 of the housing 402 falls below a predetermined threshold. In some cases, the flow control member 446 may be positioned exterior to the first cavity 416.

The first fluid outlet 418 may be configured to be coupled to an upstream irrigation supply tubing (not explicitly shown) defining a lumen for supplying water or other fluid to an endoscope. The upstream irrigation supply tube extends from a second end region (not explicitly shown) external to the housing 402 and positioned within a pump head of the peristaltic irrigation pump to a first end coupled to the first fluid outlet 418 to fluidly couple the interior of the housing 402 with the lumen of the upstream irrigation supply tube. The second end of the upstream irrigation supply tubing is configured to be fluidly coupled with an irrigation lumen of the endoscope 100. When irrigation water is required, fluid is pumped from the first cavity 416 of the housing 402 by operating the irrigation pump 315, such as by depressing a footswitch (not shown) and flows from the first reservoir 302 (arrow 452), through the first cavity 416 of the housing 402, through the first fluid outlet 418, and into the upstream irrigation supply tubing. With the flow control member 446 in the open configuration, fluid may continually flow from the fluid source (e.g., first reservoir 302) as fluid is removed from the first cavity 416 of the housing 402 for irrigation. While the first fluid outlet 418 is shown and described as adjacent to the second end region 406 of the housing 402, the first fluid outlet 418 may be positioned at other locations that are in fluid communication with the first cavity 416, as desired.

The second fluid inlet 422 may be configured to be coupled to a gas supply tubing (not explicitly shown). The gas supply tubing may extend from a first end coupled to the second fluid inlet 422 to a second end configured to be coupled to the gas/lens wash connection 290 on the outside of the connector portion 265. The gas supply tubing may define a lumen configured to supply air or an alternative gas (e.g., from the air pump 215 or an alternative gas source) to the space or second cavity 426 between the flexible sleeve 424 and the wall of the housing 402. The lumen of the gas supply tubing may be in fluid communication with the space or cavity 426 between the flexible sleeve 424 and the wall of the housing 402 via the slot 436. While the second fluid inlet 422 is shown and described as adjacent to the second end region 406 of the housing 402, the second fluid inlet 422 may be positioned anywhere along a length and/or circumference of the housing 402 which allows the second fluid inlet 422 to be in fluid communication with the cavity 426 between the inner surface of the housing 402 and the flexible sleeve 424.

A lens wash supply tubing (not explicitly shown) may be coupled to the second fluid outlet 420. The lens wash supply tubing may extend from a first end coupled to the second fluid outlet 420 to a second end configured to be coupled to the gas/lens wash connection 290 on the outside of the connector portion 265 to supply lens wash fluid to the endoscope 100. The lens wash supply tubing may define a lumen configured to supply water or fluid from the first cavity 416 of the housing 402 to the endoscope 100. While the second fluid outlet 420 is shown and described as adjacent to the second end region 406 of the housing 402, the second fluid outlet 420 may be positioned at other locations that are in fluid communication with the first cavity 416, as desired. In some embodiments, a flow control mechanism 421 may be positioned in the lens wash supply circuit. For example, a one-way valve, or other flow control mechanism 421, may be positioned at or coupled to the outlet of second fluid outlet 420 or in-line with the lens wash supply tubing. The flow control mechanism 421 may function as a back-flow prevention mechanism on the lens wash circuit and/or prevent negative back pressure potentially created during active irrigation from pulling fluid from the endoscope end backwards up the lens wash fluid conduit.

Referring additionally to FIG. 7, when lens wash is desired, the clinician may press the gas/water valve 140 downward to a second position. Air/gas is blocked from exiting the valve, allowing pressure of the air passing from the air pump 215 (or alternative gas source, if so provided) to flow through the gas supply tubing and into the space or second cavity 426 via the slot 436, as shown at arrow 460. The pressure of the gas may move the flexible sleeve 424 radially inwards, as shown at arrows 458 in FIG. 7 to increase the volume of the second cavity 426 while decreasing the volume of the first cavity 416. The pressure of the first cavity 416 may be increased as the volume thereof is decreased. The increase in the pressure of the first cavity 416 may move the flow control member 446 to block the first end opening 448 of the first fluid inlet 411 preventing additional water from entering the first cavity 416. The increase in the pressure of the first cavity 416 may push water/fluid out of the first cavity 416 through the second fluid outlet 420 and into the lens wash supply tubing, as shown at arrow 462. It is contemplated that check valves or other flow control valves positioned in-line with the irrigation supply tubing may prevent fluid from flowing through the irrigation supply tubing when the gas/water valve 140 is actuated to a second position. When the gas/water valve 140 is released, air/gas may exit the second cavity 426 and the flexible sleeve 424 may return to its original configuration creating a negative pressure in the first cavity 416. This may cause the flow control member 446 to move away from the first end opening 448 to return to an open configuration once again allowing fluid to enter the first cavity 416 from the lumen 414 of the first fluid inlet 411. In some embodiments, the housing 402 may include a bleed valve in fluid communication with the second cavity 426. The bleed valve may be actuated to purge air/gas from the second cavity 426 and allow full priming of the first cavity 416 of the housing 402.

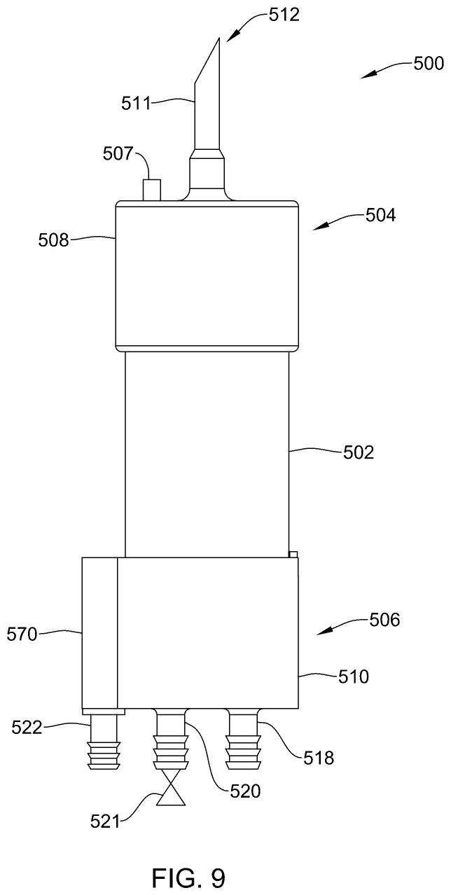

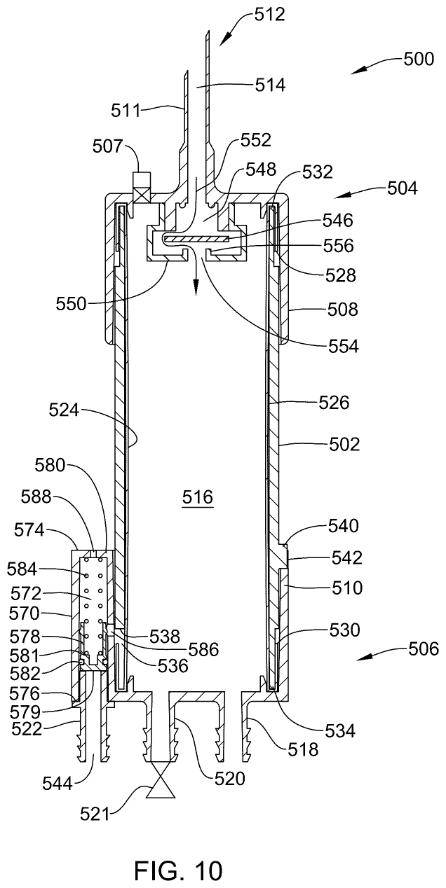

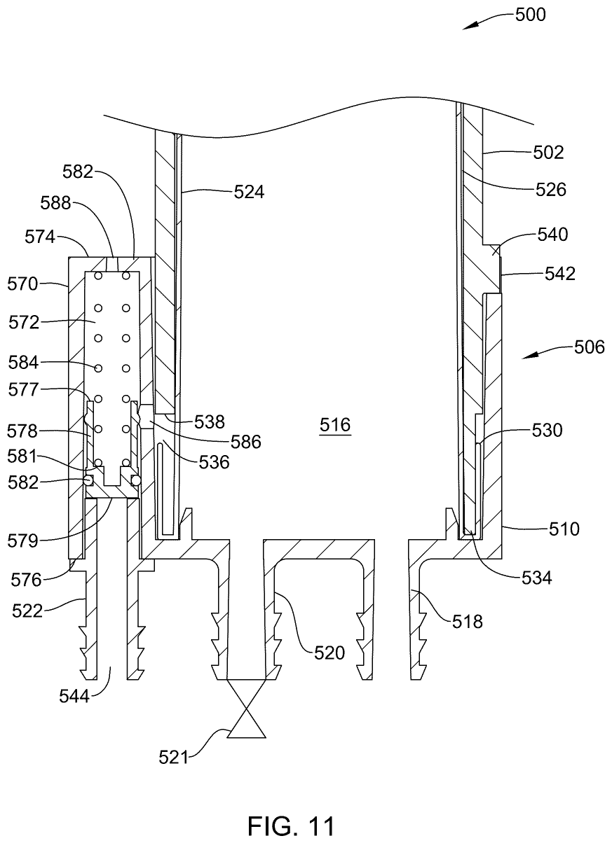

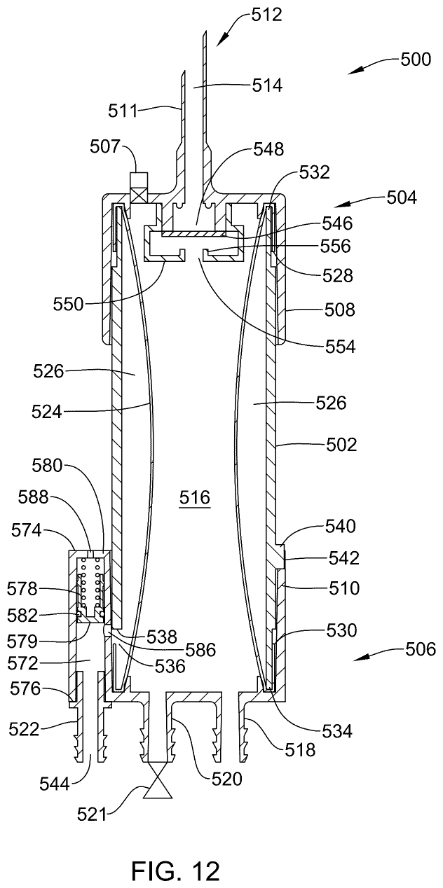

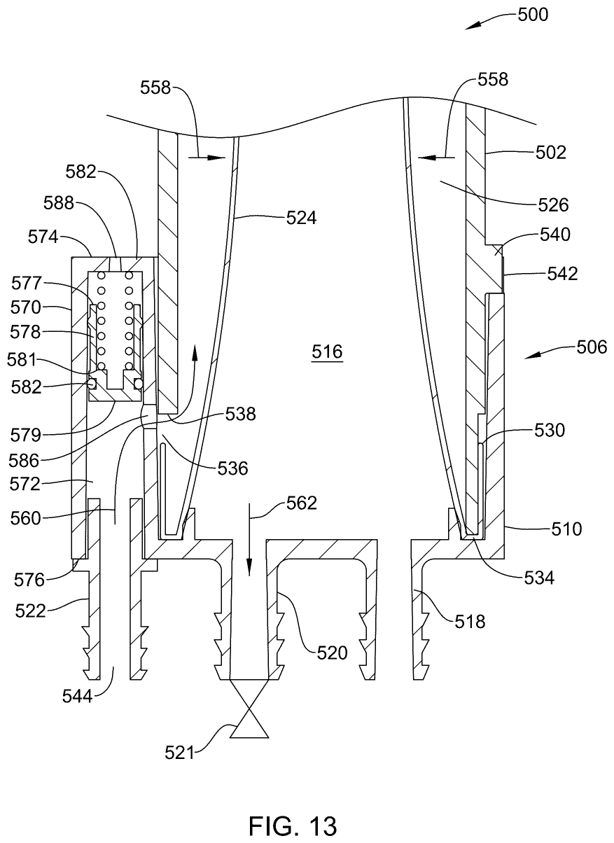

Another illustrative pressure vessel system 500 that may be used in place of the secondary fluid reservoir 330 of FIG. 3 is shown in FIGS. 9-13. FIG. 9 is a front view of the illustrative pressure vessel system 500. FIG. 10 is a cross-sectional view of the illustrative pressure vessel system 500 in an unpressurized configuration. FIG. 11 is cross-sectional view of the second end region of the illustrative pressure vessel system 500 in an unpressurized configuration. FIG. 12 is a cross-sectional view of the illustrative pressure vessel system 500 in a pressurized configuration. FIG. 13 is cross-sectional view of the second end region of the illustrative pressure vessel system 500 in a pressurized configuration. The pressure vessel system 500 may be configured to be fluidly and/or mechanically coupled to a fluid source, such as the first fluid reservoir 302 of FIG. 3. Generally, the system 500 may include a tubular housing 502 extending from a first end region 504 to a second end region 506. In some embodiments, the housing 502 may be formed from a rigid material configured to withstand the pressure required to supply the lens wash fluid. For example, the air pump 215 may be configured to supply pressurized air having a pressure in the range of about 7 to about 7.25 pounds per square inch (psi) (48.3 to about 50.0 kilopascals). Other pressure ranges may be used as desired. In some examples, the housing 502 may be formed from rigid materials such as metal, plastic, composite materials, or the like. In yet other examples, the housing 502 may be formed from a semi-rigid material.

A first lumen or cavity 516 may extend from the first end region 504 to the second end region 506 of the housing 502. In a first configuration (FIGS. 10-11), when the gas/water valve 140 is in the neutral or first position (e.g., when lens wash is not being called for), the first cavity 516 may have a volume in the range of about 90-110 cubic centimeters (cc). The volume of the first cavity 516 may be reduced when lens wash is activated (FIGS. 12-13). It is contemplated that the volume of the first cavity 516 in the first configuration may be less than 90 cc or greater than 110 cc depending on the application.

A first cap 508 may be releasably or fixedly secured to the first end region 504 and a second cap 510 may be releasably or fixedly secured to the second end region 506. The first and second caps 508, 510 may be configured to seal a first end opening and a second end opening of the housing 502, respectively. The first and second caps 508, 510 may include one or more fluid paths to selectively couple a fluid source with an endoscope 100, as will be described in more detail herein.

The first cap 508 may be generally tubular having a first generally closed end and a second end defining an opening. The opening may be sized such that the first cap 508 may be positioned over the first end region 504 of the housing 502. It is contemplated that the first cap 508 may be fixedly or releasably secured to the housing 502 in a fluid tight manner. Some illustrative coupling mechanisms may include, but are not limited to, threaded engagements, snap fits, friction fits, quick connect style couplers, adhesive or solvent bonding, thermal bonding, or the like.

The first cap 508 may include a first fluid inlet 511 extending from the first end thereof. In some embodiments, the first fluid inlet 511 may be a spike port adaptor 512. The spike port adaptor 512 may be received within the port of a fluid source. For example, the spike port adaptor 512 may be inserted directly into one of the ports 308a, 308b of the first fluid reservoir 302 of the system 300 of FIG. 3. Alternatively, in the system 300 of FIG. 3, the second outlet leg 354 of the branched connector 350 may include a spike port, similar in form and function to the ports 308a, 308b described herein, configured to receive the spike port adaptor 512. The spike port adaptor 512 may define a lumen 514 configured to fluidly couple the first fluid reservoir 302, or other fluid source, with the first cavity 516 of the housing 502 and ultimately with the lens wash supply tubing and/or irrigation supply tubing. The spike port adaptor 512 may be formed as a single monolithic structure with the first cap 508 or may be formed as a separate component subsequently coupled to the first cap 508.

In some embodiments, the first cap 508 may include a manual vent 507, such as a valve, stopcock, or the like. The manual vent 507 fluidly couples the first cavity 516 to an atmosphere outside of or exterior to the system to allow the user to vent air from the first cavity 516 during the initial setup of the endoscope system. For example, air may be purged and/or displaced from the first cavity 516 via the vent 507 to fully fill the inner cavity 516 with fluid from the water source (e.g., fluid reservoir 302). It is contemplated that the manual vent 507 may be positioned at other locations within the pressure vessel system 500, as desired. For example, the manual vent 507 may extend through the second cap 510, through the housing 502, etc.

The second cap 510 may be generally tubular having a first end defining an opening and a second generally closed end. The opening may be sized such that the second cap 510 may be positioned over the second end region 506 of the housing 502. It is contemplated that the second cap 510 may be fixedly or releasably secured to the housing 502 in a fluid tight manner. Some illustrative coupling mechanisms may include, but are not limited to, threaded engagements, snap fits, friction fits, quick connect style couplers, adhesive or solvent bonding, thermal bonding, or the like.

The second cap 510 may include a first fluid outlet 518, a second fluid outlet 520, and a second fluid inlet 522 extending from the second end thereof. In some embodiments, the second fluid inlet 522 may extend along or otherwise be coupled to a lateral side of the second cap 510. However, this is not required. In some embodiments, the second inlet 522 and/or outlets 518, 520 may be tubular ports or openings in the second cap 510 configured to selectively fluidly couple the housing 502 with another component, such as, but not limited to, a fluid or water supply tube and/or a gas supply tube. For example, the first fluid outlet 518 may be configured to be coupled with an irrigation supply tubing (similar in form and function to irrigation supply tubing 328 described above) and the second fluid outlet 520 may be configured to be coupled with a lens wash supply tubing (similar in form and function to the lens wash supply tubing 338 described above. The second fluid inlet 522 may be configured to be coupled with a gas supply tubing or an alternative gas supply tubing (similar in form and function to the gas supply tubing 336 or alternative gas supply tubing 346 described above). The second fluid inlet 522 and/or outlets 518, 520 may include hose barbs or other features configured to secure the fluid or water supply tube and/or a gas supply tube thereto.

While the first and second fluid inlets 511, 522 and the first and second fluid outlets 518, 520 are shown and described as being a part of the first and second caps 508, 510. The fluid inlets 511, 522 and/or fluid outlets 518, 520 may be formed in other parts of the pressure vessel system 500, as desired. For example, one or more of the fluid inlets 511, 522 and/or fluid outlets 518, 520 may be formed in the housing 502 of the pressure vessel system 500. It is further contemplated that while the fluid inlets 511, 522 and fluid outlets 518, 520 are shown and described as extending or protruding from the first and second caps 508, 510, the fluid inlets 511, 522 and/or fluid outlets 518, 520 may be formed as recesses or cavities. Further, as described above, the fluid inlets 511, 522 and/or fluid outlets 518, 520 may be formed as separate structures from the caps 508, 510 (or other portions of the pressure vessel system 500) and may be subsequently coupled with the caps 508, 510 (or other portions of the pressure vessel system 500) using solvent bonding, adhesives, mechanical couplings, or the like.

In some examples, the second fluid inlet 522 may be fixedly or releasably coupled to a secondary housing 570. The secondary housing 570 may be formed as a single monolithic structure with the second cap 510. However, this is not required. The secondary housing 570 may be formed as a separate structure from the second cap 510 and may be subsequently coupled thereto. The secondary housing 570 may extend from a lateral side of the second cap 510 and may have a length that is approximately equal to a length of the second cap 510. However, the secondary housing 570 may be shorter than or longer than the second cap 510, as desired.

The secondary housing 570 defines a lumen 572 extending from a first end 574 to a second end 576 thereof. In some examples, the lumen 572 may reduce in diameter at the first end 574 of the secondary housing to form a vent 588 through a top wall 580 of the secondary housing 570. A piston 578 may be movably disposed within the lumen 572. The piston 578 may be captured within the lumen 572 between a top wall 580 of the secondary housing 570 at a first end thereof and an end of the second fluid inlet 522 at a second end thereof. The piston 578 may include a gasket, O-ring, or other sealing member 582 extending about a perimeter thereof. The sealing member 582 may fluidly isolate a first end 577 of the piston 578 from a second end 579 of the piston 578. Alternatively, or additionally, the piston 578 may be molded from an elastomeric material as a single monolithic structure that incorporates both the piston and the sealing elements. This may reduce or eliminate the need for one or more O-rings or other seals.

A biasing member 584, such as, but not limited to, a spring, may be positioned within the lumen 572. The biasing member 584 may extend between an intermediate region 581 of the piston 578 (or a first end 577 of the piston 578) and an inner surface of the top wall 580 of the secondary housing 570. The biasing member 584 may be configured to bias the piston 578 towards the second end 576 of the secondary housing 570 in the absence of an external biasing force. The piston 578 may take a number of different configurations. For example, in the illustrated embodiment, the piston 578 is substantially tubular with variable inner and outer diameter and an enclosed second end 579. The enclosed second end 579, in combination with the sealing member 582 may selectively fluidly isolate the second fluid inlet 522 and an interior of the housing 502. In other examples, the piston 578 may be substantially solid (e.g., a generally cylindrical component). A port 586 may extend through a lateral sidewall of the secondary housing 570. The port 586 selectively fluidly couples the lumen 544 of the second fluid inlet 522 with an interior of the housing 502 via the lumen 572 of the secondary housing 570. For example, the piston 578 may be biased towards the first end 574 of the secondary housing 570 to selectively fluidly couple the second fluid inlet 522 and an interior of the housing 502.

The pressure vessel system 500 may further include an elastomeric or flexible sleeve 524 disposed within the first cavity 516 of the housing 502. The flexible sleeve 524 may have a generally tubular configuration and may be positioned along the inner surface of the housing 502 (e.g., within the first cavity 516). The flexible sleeve 524 may be free from attachment to the housing 502 along at least a portion of the length of the flexible sleeve 524 to define a variable volume space or second cavity 526 between the inner surface of the wall of the housing 502 and the flexible sleeve 524. The flexible sleeve 524 may be formed from a flexible or deformable material that can be stretched and subsequently return to an original configuration. For example, in response to an increase in air/gas volume in the space or second cavity 526 between the wall of the housing 502 and the flexible sleeve 524, the flexible sleeve 524 may extend radially inwards reducing the volume of the first cavity 516. As air is evacuated, the second cavity 526 and the volume thereof reduces, the flexible sleeve 524 may return to its original configuration along the inner surface of the wall of the housing 502. In some embodiments, the flexible sleeve 524 may be formed from a thin wall silicone, rubber, thermoplastic elastomer (TPE), or other elastomeric material which can be stretched to create tensile loading and capable of re-coiling after being strained in the plastic deformation region of the stress-strain curve. The flexible sleeve 524 may take many forms. In the illustrative example, the flexible sleeve 524 may be cylindrical and positioned along an inside wall of the tubular housing 502, thus allowing pressurization from all sides and facilitating a larger displacement efficiency. In other examples, the flexible sleeve 524 may extend less than 360° about an inner circumference of the tubular housing 502. When the flexible sleeve 524 extends less than 360°, the lateral edges of the flexible sleeve 524 may be secured along the length of the sleeve 524 to the inner surface of the housing 502 to form an air-tight seal.

The flexible sleeve 524 may extend from a first end 528 to a second end 530. In some examples, the first end 528 may extend beyond a first end 532 of the housing 502 and fold over to extend along an outer surface of the housing 502. The first end 528 of the flexible sleeve 524 may be secured between an inner surface of the first cap 508 and the outer surface of the housing 502. In some examples, the second end 530 of the flexible sleeve 524 may extend beyond a second end 534 of the housing 502 and fold over to extend along an outer surface of the housing 502. The second end 530 of the flexible sleeve 524 may be secured between an inner surface of the second cap 510 and the outer surface of the housing 502. The wall thickness of the housing 502 may be reduced or thinned for a length adjacent to the first end 532 and/or the second end 534 of the housing 502. This may allow the flexible sleeve 524 to be more easily mechanically captured between the caps 508, 510 and the outer surface of the housing 502. However, this is not required. In some embodiments, the housing 502 may have a uniform wall thickness along an entire length thereof. In some cases, the flexible sleeve 524 may be additionally or alternatively coupled to the housing 502 using a number of techniques such as adhesive or solvent bonding, thermal bonding, and the like. In another embodiment, the flexible sleeve 524 may transition from a thin wall to a thick ring at one or both ends 528, 530. The thick ring(s) may function as a gasket between the rigid housing 502 and one or both caps 508, 510. It is further contemplated that the flexible sleeve 524 may have a length that is less than a length of the housing 502. For example, an end 528, 530 of the flexible sleeve 524 may be secured at a location between the first and second end 532, 534 of the housing 502.

The housing 502 may further include a slot 536 extending from the second end 534 thereof towards the first end 532 thereof. In some cases, the slot or opening 536 may be spaced from the second end 534 of the housing 502. A first end 538 of the slot 536 may be positioned between the first end 574 and the second end 576 of the secondary housing 570. In some cases, the first end 538 of the slot 536 may be adjacent to or above the port 586 of the secondary housing 570 such that the slot 536 is fluidly coupled to the port 586. Further, the slot 536 may have a width less than a width of the secondary housing 570 such that the slot 536 is in selective fluid communication with a lumen 544 of the second fluid inlet 522 but does not extend beyond the secondary housing 570. The slot 536 may selectively fluidly couple a lumen 544 of the second fluid inlet 522 with the second cavity 526 via the lumen 572 of the secondary housing 570. Thus, pressurized air/gas may flow from the air pump 215 or alternative gas source through the second fluid inlet 522, through the lumen 572 of the secondary housing 570, through the port 586, and into the second cavity 526 to fill/pressurize the second cavity 526. In some examples, the housing 502 and the second cap 510 may include mating alignment features configured to facilitate assembly of the second cap 510 with the housing 502 such that the port 586 aligns with the slot 536. For example, the housing 502 may include a protrusion 540 and the second cap 510 may include a mating slot 542. Other alignment mechanisms or visual indicia may be used as desired.

A flow control member 546 may be positioned within the first cavity 516 of the housing 502 adjacent to the first fluid inlet 511. The flow control member 546 may be configured to selectively block or occlude an opening 548 of the first fluid inlet 511. When the flow control member 546 is disposed against or over the opening 548 of the first fluid inlet 511, fluid is prevented from flowing from the fluid source (e.g., first reservoir 302) into the first cavity 516 of the housing 502 (see, for example, FIG. 12) and from flowing from the first cavity 516 of the housing 502 into the fluid source. In some cases, the flow control member 546 may be a floating stopper held against the first end opening 548 of the first fluid inlet 511. The flow control member 546 may be disposed against or over the first end opening 548 of the first fluid inlet 511 by an increase in the pressure in the first cavity 516 within the housing 502. When the pressure is vented (e.g., by releasing the gas/water valve 140), the pressure in the first cavity 516 of the housing 502 may decrease allowing the flow control member 546 to be displaced away from first end opening 548 of the first fluid inlet 511 such that fluid can flow into the first cavity 516 of the housing 502, as shown at arrow 552. A stop mechanism 550 may be positioned adjacent to the flow control member 546 to prevent the flow control member 546 from dropping to a bottom of the housing 502. In some examples, the flow control member 546 may contact an upper edge of the stop mechanism 550 to stop a flow of fluid through the first fluid inlet 511. The stop mechanism 550 may include one or more openings 554 that are smaller than the flow control member 546 to allow fluid to pass while maintaining the flow control member 546 adjacent to the opening 548 of the first fluid inlet 511. The flow control member 546 may be spaced from the opening 554 by one or more circumferentially spaced ribs 556 extending from the stop mechanism 550. The one or more circumferentially spaced ribs 556 may have gaps or openings therebetween to allow fluid to pass between the flow control member 546 and the stop mechanism 550. In other examples, the flow control member 546 may be a one-way valve that allows fluid to flow into the first cavity 516 of the housing 502 when the pressure of the first cavity 516 of the housing 502 falls below a predetermined threshold. In some cases, the flow control member 546 may be positioned exterior to the first cavity 516.

The first fluid outlet 518 may be configured to be coupled to an upstream irrigation supply tubing (not explicitly shown) defining a lumen for supplying water or other fluid to an endoscope. The upstream irrigation supply tube extends from a second end region (not explicitly shown) external to the housing 502 and positioned within a pump head of the peristaltic irrigation pump to a first end coupled to the first fluid outlet 518 to fluidly couple the interior of the housing 502 with the lumen of the upstream irrigation supply tube. The second end of the upstream irrigation supply tubing is configured to be fluidly coupled with an irrigation lumen of the endoscope 100. When irrigation water is required, fluid is pumped from the first cavity 516 of the housing 502 by operating the irrigation pump 315, such as by depressing a footswitch (not shown) and flows from the first reservoir 302 (arrow 552), through the first cavity 516 of the housing 502, through the first fluid outlet 518, and into the upstream irrigation supply tubing. With the flow control member 546 in the open configuration, fluid may continually flow from the fluid source (e.g., first reservoir 302) as fluid is removed from the first cavity 516 of the housing 502 for irrigation. While the first fluid outlet 518 is shown and described as adjacent to the second end region 506 of the housing 502, the first fluid outlet 518 may be positioned at other locations that are in fluid communication with the first cavity 516, as desired.

The second fluid inlet 522 may be configured to be coupled to a gas supply tubing (not explicitly shown). The gas supply tubing may extend from a first end coupled to the second fluid inlet 522 to a second end configured to be coupled to the gas/lens wash connection 290 on the outside of the connector portion 265. The gas supply tubing may define a lumen configured to supply air or an alternative gas (e.g., from the air pump 215 or an alternative gas source) to the space or second cavity 526 between the flexible sleeve 524 and the wall of the housing 502. The lumen of the gas supply tubing may be in selective fluid communication with the space or cavity 526 between the flexible sleeve 524 and the wall of the housing 502 via the slot 536. While the second fluid inlet 522 is shown and described as adjacent to the second end region 506 of the housing 502, the second fluid inlet 522 may be positioned anywhere along a length and/or circumference of the housing 502 which allows the second fluid inlet 522 to be in selective fluid communication with the cavity 526 between the inner surface of the housing 502 and the flexible sleeve 524.

A lens wash supply tubing (not explicitly shown) may be coupled to the second fluid outlet 520. The lens wash supply tubing may extend from a first end coupled to the second fluid outlet 520 to a second end configured to be coupled to the gas/lens wash connection 290 on the outside of the connector portion 265 to supply lens wash fluid to the endoscope 100. The lens wash supply tubing may define a lumen configured to supply water or fluid from the first cavity 516 of the housing 502 to the endoscope 100. While the second fluid outlet 520 is shown and described as adjacent to the second end region 506 of the housing 502, the second fluid outlet 520 may be positioned at other locations that are in fluid communication with the first cavity 516, as desired. In some embodiments, a flow control mechanism 521 may be positioned in the lens wash supply circuit. For example, a one-way valve, or other flow control mechanism 521, may be positioned at or coupled to the outlet of second fluid outlet 520 or in-line with the lens wash supply tubing. The flow control mechanism 521 may function as a back-flow prevention mechanism on the lens wash circuit and/or prevent negative back pressure potentially created during active irrigation from pulling fluid from the endoscope end backwards up the lens wash fluid conduit.

Referring additionally to FIGS. 12-13, when lens wash is desired, the clinician may press the gas/water valve 140 downward to a second position. Air/gas is blocked from exiting the valve, allowing pressure of the air passing from the air pump 215 (or alternative gas source, if so provided) to flow through the gas supply tubing and into the lumen 572 of the secondary housing 570. The pressure of the air/gas may overcome the force of the biasing member 584 to bias or move the piston 578 towards the first end 574 of the secondary housing 570. Once the second end 579 of the piston 578 is advanced beyond the port 586, air/gas may flow from the lumen 544 of the second fluid inlet 522, through the lumen 572 of the secondary housing 570, through the port 586, and into the second cavity 526 via the slot 536, as shown at arrow 560. It is contemplated that the port 586 may be sized such that only a portion of the air/gas is diverted into the second cavity 526 leaving sufficient air/gas in the lumen 572 to maintain the piston 578 in the actuated or biased configuration. The pressure of the gas may move the flexible sleeve 524 radially inwards, as shown at arrows 558 in FIG. 13 to increase the volume of the second cavity 526 while decreasing the volume of the first cavity 516. The pressure of the first cavity 516 may be increased as the volume thereof is decreased. The increase in the pressure of the first cavity 516 may move the flow control member 546 to block the first end opening 548 of the first fluid inlet 511 preventing additional water from entering the first cavity 516. The increase in the pressure of the first cavity 516 may push water/fluid out of the first cavity 516 through the second fluid outlet 520 and into the lens wash supply tubing, as shown at arrow 562. It is contemplated that check valves or other flow control valves positioned in-line with the irrigation supply tubing may prevent fluid from flowing through the irrigation supply tubing when the gas/water valve 140 is actuated to a second position. When the gas/water valve 140 is released, air/gas may exit the second cavity 526 and the flexible sleeve 524 may return to its original configuration creating a negative pressure in the first cavity 516. For example, when the gas/water valve 140 is released, the biasing member 584 may return to an original elongated configuration biasing or moving the piston 578 towards the second end 576 of the secondary housing 570. The second cavity 526 may remain in fluid communication with the lumen 572 of the secondary housing 570 (via the slot 536 and port 586). Air/gas may exit the lumen 572 via the vent 588. The decrease in pressure of the first cavity 516 may cause the flow control member 546 to return to an open configuration once again allowing fluid to enter the first cavity 516 from the lumen 514 of the first fluid inlet 511. In some embodiments, the vent 588 may be omitted (e.g., the top wall 580 may be solid) and the air/gas may vent via the gas supply tubing.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a first container configured to contain a fluid, the first container having a first port in fluid communication with a bottom portion thereof; and
a pressure vessel system, the pressure vessel system comprising:
a housing defining a first cavity;
a sleeve disposed within the first cavity of the housing and defining a second cavity between an inner surface of a wall of the housing and the sleeve;
a first fluid inlet in selective fluid communication with the first cavity;
a second fluid inlet in fluid communication with the second cavity; and
a first fluid outlet in fluid communication with the first cavity.

2. The container and tube set of claim 1, further comprising a first cap secured to a first end of the housing.

3. The container and tube set of claim 2, wherein the first fluid inlet is formed in the first cap.

4. The container and tube set of claim 2, further comprising a second cap secured to a second end of the housing.

5. The container and tube set of claim 4, wherein the first fluid outlet and the second fluid inlet are formed in the second cap.

6. The container and tube set of claim 4, further comprising a vent in one of the first or second caps, the vent fluidly coupling the first cavity with an atmosphere outside the system.

7. The container and tube set of claim 1, further comprising a first flow control member positioned adjacent to the first fluid inlet.

8. The container and tube set of claim 1, further comprising a secondary housing fluidly coupled with the second fluid inlet, the secondary housing defining a lumen.

9. The container and tube set of claim 8, further comprising a piston disposed within the secondary housing.

10. The container and tube set of claim 9, wherein the piston is actuatable to selectively couple the second fluid inlet with the second cavity.

11. The container and tube set of claim 9, further comprising a biasing member disposed between an end of the piston and an end of the secondary housing, the biasing member configured to bias the piston towards the second fluid inlet.

12. The container and tube set of claim 1, further comprising a flow control mechanism fluidly coupled to the first fluid outlet.

13. The container and tube set of claim 1, wherein the sleeve is a flexible tubular member.

14. The container and tube set of claim 1, further comprising a slot extending through a sidewall of the housing, the slot configured to be in fluid communication with the second fluid inlet.

15. The container and tube set of claim 1, wherein a volume of the second cavity is configured to selectively increase to increase a pressure of the first cavity.

16. A container and tube set arranged and configured to couple to an endoscope for use in an endoscopic procedure, the container and tube set comprising:
a first container configured to contain a fluid, the first container having a first port in fluid communication with a bottom portion thereof; and
a pressure vessel system, the pressure vessel system comprising:
a generally tubular housing extending from a first end to a second end and defining a first cavity;
a first cap coupled with the first end of the generally tubular housing;
a second cap coupled with the second end of the generally tubular housing;
a sleeve extending from a first end to a second end and disposed within the first cavity of the housing and defining a second cavity between an inner surface of a wall of the housing and the sleeve;
a first fluid inlet in selective fluid communication with the first cavity;
a second fluid inlet in fluid communication with the second cavity; and
a first fluid outlet in fluid communication with the first cavity;
wherein the first end of the sleeve is positioned between an outer surface of the generally tubular housing and the first cap and the second end of the sleeve is positioned between the outer surface of the generally tubular housing and the second cap.

17. The container and tube set of claim 16, wherein a volume of the second cavity is configured to selectively increase to increase a pressure of the first cavity.

18. A container and tube set arranged and configured to couple to an endoscope for use in an endoscopic procedure, the container and tube set comprising:
a first container configured to contain a fluid, the first container having a first port in fluid communication with a bottom portion thereof; and
a pressure vessel system, the pressure vessel system comprising:
a generally tubular housing extending from a first end to a second end and defining a first cavity;
a first cap coupled with the first end of the generally tubular housing;
a second cap coupled with the second end of the generally tubular housing;
a secondary housing extending from a lateral side of the second cap, the secondary housing defining a lumen;
a piston disposed within the lumen of the secondary housing;
a sleeve extending from a first end to a second end and disposed within the first cavity of the housing and defining a second cavity between an inner surface of a wall of the housing and the sleeve;
a first fluid inlet in selective fluid communication with the first cavity;
a second fluid inlet in fluid communication with the second cavity; and
a first fluid outlet in fluid communication with the first cavity.

19. The container and tube set of claim 18, wherein the piston is actuatable to selectively couple the second fluid inlet with the second cavity.

20. The container and tube set of claim 18, further comprising a biasing member disposed between an end of the piston and an end of the secondary housing, the biasing member configured to bias the piston towards the second fluid inlet.
